(19) United States
(12) Patent Application Publication
(10) Pub. No.: US 2021/0060296 A1
(43) Pub. Date: Mar. 4, 2021

(54) MINIATURIZED INTRA-BODY CONTROLLABLE MEDICAL DEVICE

(71) Applicant: Miraki Innovation Think Tank, LLC, CAMBRIDGE, MA (US)

(72) Inventors: Christopher J. Velis, Cambridge, MA (US); Matthew P. Palmer, Medford, MA (US)

(21) Appl. No.: 17/042,658

(22) PCT Filed: Mar. 27, 2019
(86) PCT No.: PCT/US2019/024247

§ 371 Date: Sep. 28, 2020

Related U.S. Application Data
(60) Provisional application No. 62/649,490, filed on Mar. 28, 2018.

(51) Int. Cl. A61M 25/01; A61B 8/12; A61B 5/055; A61B 1/04; A61B 5/00; A61B 5/01; A61B 6/00

(52) U.S. Cl. CPC A61M 25/0116; A61B 8/12; A61B 5/055; A61B 1/041; A61N 5/1002; A61B 5/015; A61B 5/0084; A61B 5/0035; A61B 6/425; A61B 5/0066

(57) ABSTRACT

A medical device includes a host structure that has an interior area and one or more propulsion systems linked to the host structure. The host structure and the propulsion systems are configurable into a peripheral boundary of a size adapted to fit in a lumen or cavity of a living organism such as a human being or animal. The medical device includes one or more power supplies in communication with the propulsion systems. The medical device includes a control unit in communication with the propulsion systems and the power supplies. The control unit has a computer process controller configured to control the propulsion systems to move the host structure and the propulsion systems in the lumen so that the host structure and the propulsion systems are self-maneuverable within the lumen.

### FIELD OF THE INVENTION

The present invention relates generally to a miniaturized intra-body controllable medical device. More specifically, the invention relates to the intra-body medical device having a propulsion system, a deployment system, a control system, a power supply system, an intra-device storage system, an imaging system, a therapy system, a sample and data gathering system, and/or a material dispensing system. Furthermore, the invention details materials for an intra-body controllable medical device, an interactive group of intra-body controllable medical devices, configurations for intra-body controllable medical devices, and methods of using intra-body controllable medical devices.

This disclosure relates to miniaturized intra-body controllable medical devices. These may be externally controllable or may be fully autonomous. These may communicate via a tether or may communicate wirelessly. The intra-body medical device may have a propulsion system, a deployment system, a control system, a power supply system, an intra-device storage system, an imaging system, a therapy system, a sample and data gathering system, and/or a material dispensing system. The devices may work independently or work together in a group.

### BACKGROUND OF THE INVENTION

Many medical procedures require the physician to gain access to regions within the body in order to complete a diagnosis or provide therapy to a patient. Often, physicians access internal regions of the body through the body's own natural orifices and lumens. Natural orifices include the nostrils, mouth, ear canals, nasolacrimal ducts, anus, urinary meatus, vagina, and nipples. The lumens include the interior of the gastrointestinal tract, the pathways of the bronchi in the lungs, the interior of the renal tubules and urinary collecting ducts, the pathways of the vagina, uterus, and fallopian tubes. From within these orifices and lumens, physicians can create an incision to gain access to almost any region of the body.

Traditional methods for gaining access to regions within the body include open surgical procedures, laparoscopic procedures and endoscopic procedures. Laparoscopic procedures allow the physician to use a small “key-hole” surgical opening and specially designed instruments to gain access to regions within the body. Initially, laparoscopic instruments were linear in nature, and required a straight obstruction free “line-of-sight” to access regions of the body. Endoscopic procedures allow the physician to access regions of the digestive system by passing flexible instruments through either the mouth or rectum.

Recently, physicians have begun to control these instruments using robots. These robots are typically connected in master/slave configuration, where the robot translates the physician's movements into instrument movements. Robotic controls have also allowed for advent of flexible laparoscopic instruments. Medical robots still require a physician to be actively controlling the movements and actions of the devices being controlled and require large expensive capital equipment and dedicated operating room spaces.

Additionally, pill capsules have been invented that allow for a patient to ingest the capsule and as it passes through the digestive system takes pictures. There are no means for: controlling the motion of these devices, tracking or controlling the orientation, speed or location of these devices, accurately knowing where pictures were taken, and performing any type of surgical procedure or delivering therapy.

Thus, improvements are desirable in this field of technology. It would be beneficial to combine the ability to perform surgical procedures and provide therapy using robotic instruments with the footprint, size, and maneuverability of capsule systems or other structures. It would be beneficial to provide a means for controlling the movement of a medical device so that the surgeon can navigate it to a specific location.

### SUMMARY

There is disclosed herein a medical device for intra-body conveyance. The medical device includes a host structure that has an interior area and one or more propulsion systems linked to the host structure. The host structure and the propulsion systems are configurable into a peripheral boundary of a size adapted to fit in a lumen or cavity of a living organism such as a human being or animal. The medical device includes one or more power supplies in communication with the propulsion systems. The medical device includes a control unit in communication with the propulsion systems and the power supplies. The control unit has a computer process controller configured to control the propulsion systems to move the host structure and the propulsion systems in the lumen so that the host structure and the propulsion systems are self- maneuverable within the lumen.

The propulsion systems include one or more of a sprocket driven track structure in communication with the host structure; a fluid jet stream discharging from the host structure; a plurality of articulating tentacles extending from the host structure; a screw-drive formed on external surfaces of the host structure; a pull device and/or a push device in communication with the host structure; and an arrangement of inflating and deflating balloons, the balloons are in predetermined positions on the host structure and/or in predetermined positions around the host structure.

In one embodiment, the propulsion system includes an orientation control device configured for orientation control of the medical device within the lumen. In one embodiment, the orientation control devices and/or the propulsion systems include one or more of stabilization wings, flippers, anchors, braces, supports, clamps, and a gyroscope and ballast systems.

In one embodiment, the medical device includes one or more docking stations for receiving a tether, a medical scope and/or a second medical device. The medical scope may be an ENT otoscope, a naso-pharyngoscope, a laparoscope, a sinuscope, a colposcope, a resectoscope and a cystoscope.

In one embodiment, the docking station includes the tether, a holding device, a release device, a launch device, a push device and/or a pull device.

In one embodiment, the medical device includes a tracking device, a signal transmitter, and/or a signal receiver in communication with the control unit for tracking and guiding the medical device within the lumen.

The power supplies may include miniaturized batteries, fuel cells, electrochemical reactors, piezoelectric devices, energy harvesting devices that obtains thermal and/or chemical reaction energy from the fluids in and tissue of the lumen and adjacent organs, thermal reactors, heat absorption energy conversion devices and triboelectric energy harvesting devices.

In one embodiment, the host structure includes one or more storage systems that have miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables.

The host structure may include or be manufactured from a clinically inert material, a sterilizable material, an elastomeric material, a chemically reactive material, a chemically inert material, a disintegrable material, a dissolvable material, a collapsible material and a material having physical and chemical properties to withstand exposure to bodily fluids for predetermined periods of time.

The host structure may include one or more imaging systems, such as X-ray radiography imaging, magnetic resonance imaging, medical ultrasonography or ultrasound, confocal microscopy, elastography, optical-coherence tomography, tactile imaging, thermography and medical digital photography. In one embodiment, the imaging system is configured to travel through the lumen in the medical device. In one embodiment, the imaging system is configured to be discharged from the medical device while in the lumen and deposited in a predetermined location in the lumen for ongoing monitoring.

The host structure may include a therapy delivery system, such as an optical-coherence tomography (OCT) guided laser instruments, radiation discharging sources, chemotherapy deploying devices, pharmaceutical and drug deploying devices, and photodynamic therapy devices. In one embodiment, the therapy delivery system is configured to travel through the lumen in the medical device and provide therapy. In one embodiment, the therapy delivery system is configured to be discharged from the medical device while in the lumen and deposited in a predetermined location in the lumen for ongoing therapy delivery. In one embodiment, the therapy delivery system is configured with a storage medium configured to record at least one of record time, duration and application location of the therapy.

In one embodiment, the host structure includes a sample gathering system and/or a data gathering system. In one embodiment, the sample gathering system is configured to obtain at least one of tissue biopsies and blood, bone, cells, bone marrow, blood, urine, DNA and fecal samples. In one embodiment, the data gathering devices includes one or more of pH probes, accelerometers, pressure transducers, thermometers, and dimensional measurement systems.

In one embodiment, the host structure includes one or more material dispensing systems equipped with at least one storage compartment configured for storing and dispensing payloads. In one embodiment, the payloads store or hold medication, liquids, powders, chemically reactive agents and radiation emitting sources. The material dispensing systems may include one or more of an actuator, a pump (240Q), a compressor, a nozzle, a flow control device, an injection device, a piercing device a dose measuring device and a recording device.

The present invention includes an interactive group of two or more of the foregoing the medical devices. The interactive group of the medical devices are in communication with at least one of an external computer-based control system and each other and are configured to cooperate with each another to perform at least one predetermined mission.

The present invention includes a method for using the foregoing medical devices for administering medications, administering therapy, deploying medical devices, imaging and/or surgery.

The present invention includes methods for using the foregoing medical devices for use in a gastro/intestinal tract, use in urology applications, use in a lung, use in a bladder, use in a nasal system, use in a reproductive system, use in performing Transurethral Resection of Bladder Tumors (TURBT), use in Transurethral Resection of the Prostate (TURP), use in trans rectal prostate ultrasound, biopsy, and/or radiation treatment.

The present invention includes methods for using the forgoing medical devices in procedural environments, operatory and surgical procedures, ambulatory and out-patient procedures and/or unobtrusive normal routine living.

The present invention includes a plurality of medical devices in communication with at least one repository such as a heat sink, a chemical reactor and a storage vessel. The plurality of medical devices may include a cooling system and a material discharge system. The repository may be positioned intra body and outside the body.

### DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A illustrates a representative intra-body controllable medical device formed in accordance with the present invention.

FIG. 1B illustrates a representative intra-body controllable medical device formed in accordance with the present invention.

FIG. 2 illustrates an alternative representation of an intra-body controllable medical device formed in accordance with the present invention.

FIG. 3 illustrates an intra-body controllable medical device featuring a helical screw drive propulsion system formed in accordance with the present invention.

FIG. 4 illustrates an intra-body controllable medical device featuring a sprocket driven track propulsion system formed in accordance with the present invention.

FIG. 5 illustrates an alternative representation of an intra-body controllable medical device featuring a sprocket driven track propulsion system formed in accordance with the present invention.

FIG. 6 illustrates an intra-body controllable medical device featuring a fluid/jet stream propulsion system formed in accordance with the present invention.

FIG. 7 illustrates an intra-body controllable medical device featuring a tentacle propulsion system formed in accordance with the present invention.

FIG. 8 illustrates an alternative representation of an intra-body controllable medical device featuring a tentacle propulsion system formed in accordance with the present invention.

FIGS. 9A, 9B and 9C illustrate an intra-body controllable medical device featuring an anchor and tether propulsion system formed in accordance with the present invention.

FIGS. 9D and 9E illustrate an intra-body controllable medical device featuring a push type propulsion system formed in accordance with the present invention.

FIGS. 9F and 9G illustrate an intra-body controllable medical device featuring magnetic field type propulsion system formed in accordance with the present invention.

FIGS. 10A, 10B and 10C illustrate an intra-body controllable medical device featuring an inflating/deflating balloon propulsion system formed in accordance with the present invention.

FIG. 11 illustrates an intra-body controllable medical device featuring gyroscopic and wing/flipper stabilization systems formed in accordance with the present invention.

FIGS. 12A-G illustrate different scope systems for deploying an intra-body controllable medical device.

FIGS. 13A, 13B and 13C illustrate an intra-body controllable medical device being deployed by a scope.

FIGS. 14A and 14B illustrate an intra-body controllable medical device being deployed into the stomach by a scope.

FIGS. 15A and 15B illustrate systems for controlling an intra-body controllable medical device.

FIGS. 16A and 16B illustrate different power supply systems for powering an intra-body controllable medical device.

FIG. 17 illustrates the use of induction charging to power an intra-body controllable medical device.

FIG. 18 illustrates tethered power transfer between two intra-body controllable medical devices.

FIG. 19 illustrates intra-device storage systems for intra-body controllable medical devices.

FIGS. 20A and 20B illustrate imaging systems that can be incorporated with an intra-body controllable medical device.

FIGS. 21A and 21B illustrates the placement of a monitoring sensor by an intra-body controllable medical device.

FIGS. 22A, 22B and 22C illustrate the delivery of a therapy system by an intra-body controllable medical device.

FIGS. 23A-D illustrate different tissue and fluid sampling devices that may be used by an intra-body controllable medical device.

FIG. 24 illustrates material dispensing systems that may be used by an intra-body controllable medical device.

FIG. 25 illustrates an interactive group of intra-body medical devices.

FIG. 26 illustrates another interactive group of intra-body medical devices.

### DETAILED DESCRIPTION

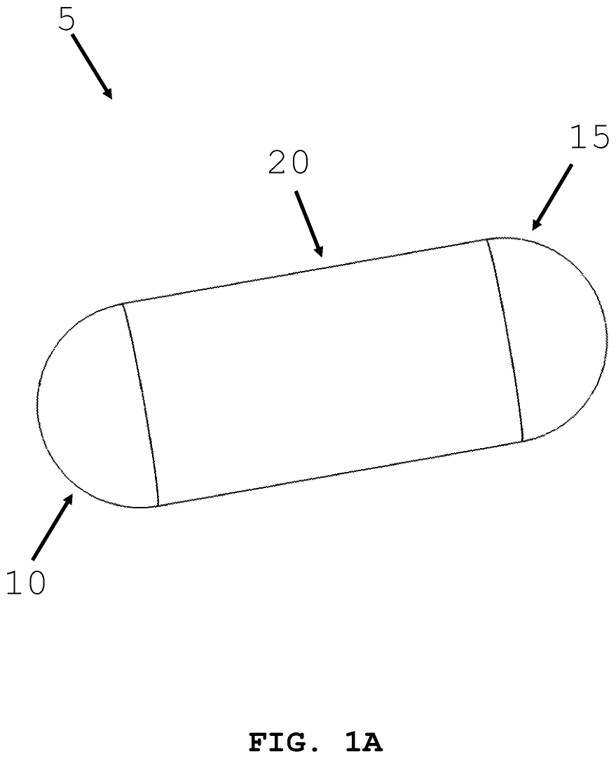

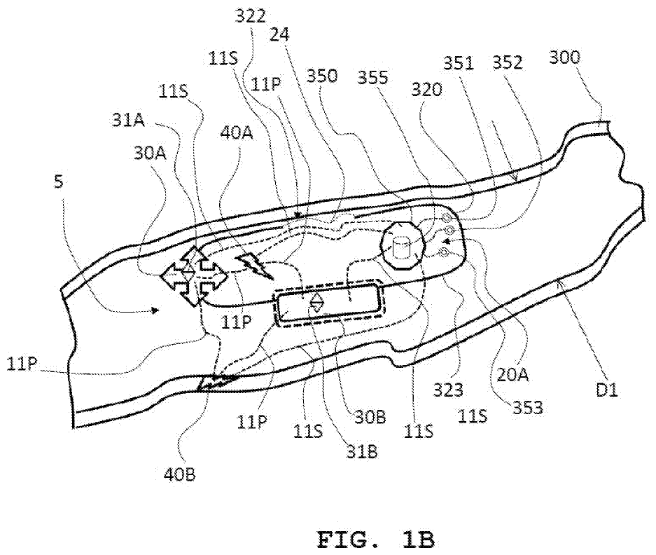

FIG. 1A illustrates an exemplary intra-body controllable medical device (hereinafter “the medical devices”). In one embodiment, the intra-body controllable medical device 5 is capsule shaped. Intra-body controllable medical device 5 has a distal end 10, a proximal end 15, and body 20 connecting the distal end 10 and proximal end 15. A control unit, a power supply system, an intra-device storage system, an imaging system, a therapy system, a sample and data gathering system, and a material dispensing system may be located within body 20 of the medical device 5, as described herein. The intra-body controllable medical device may be sized according to the anatomy that it will need to navigate, and the method used to deliver it. As an For example, overall dimensions for an intra-body controllable device operating within the gastrointestinal track may have a diameter of about 25 mm and a length of about 75 mm. More preferably, the device may have a diameter of about 15 mm and a length of about 50 mm. Most preferably, the diameter may be less than about 15 mm and a length of less than about 50 mm. Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15 mm and the length less than 50 mm. Control system, power supply system, intra-device storage system, imaging system, therapy system, sample and data gathering system, and material dispensing systems are sized to fit within these dimensional guidelines.

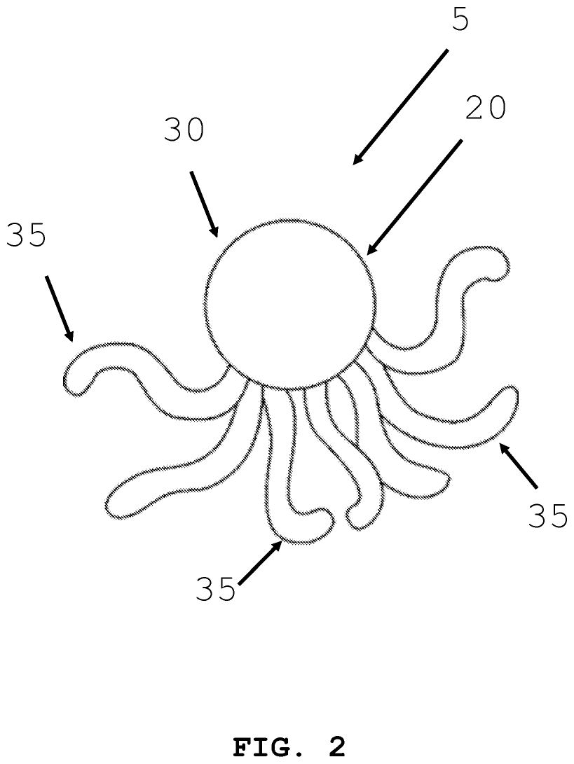

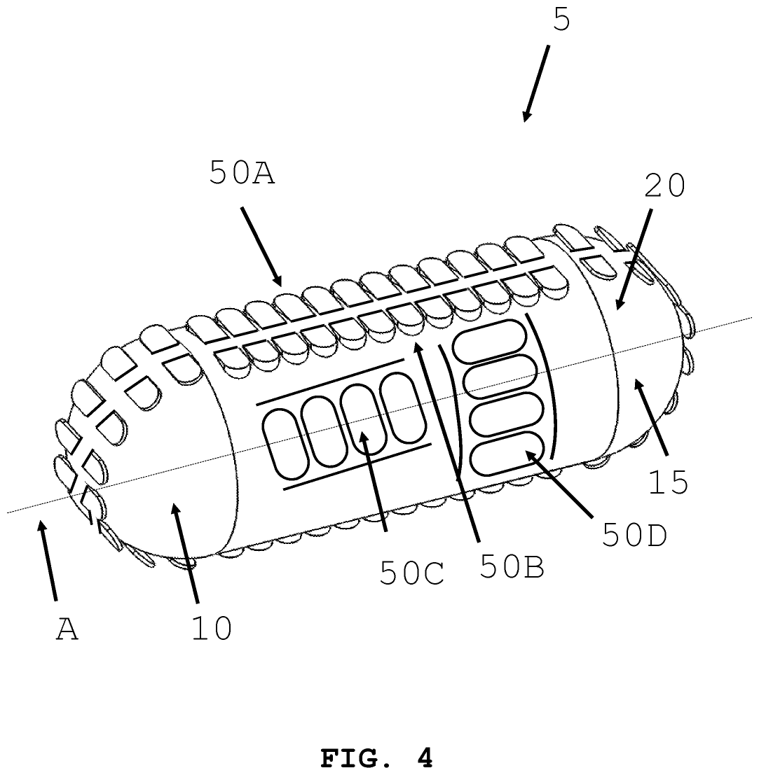

As shown in FIG. 1B, the medical device 5 includes the body 20 which is a host structure 320 that has an interior area 20A. A first propulsion system 30A and a second propulsion system 30B (e.g., a sprocket and track system similar to those shown and described with reference to FIGS. 4 and 5) are linked to the host structure 320. While the first propulsion system 30A and a second propulsion system 30B are shown and described, the present invention is not limited in this regard as only one propulsion system or more than two propulsion systems may be employed without departing from the broader aspects of the present invention. The first propulsion system 30A (e.g. see FIGS. 2-8) and the second propulsion system 30B are configurable into a peripheral boundary 323 (e.g., a skin or exterior surface) of a miniaturized size and are adapted to fit in a lumen 300 (or tissue, muscle or fat) of a living organism, such as a human. In one embodiment, the medical device 5 is configured to navigate in bone marrow within a bone. As an example, overall dimensions for an intra-body controllable device operating within the gastrointestinal track may have a diameter of about 25 mm and a length of about 75 mm. More preferably, the device may have a diameter of about 15 mm and a length of about 50 mm. Most preferably, the diameter may be less than about 15 mm and a length of less than about 50 mm. Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15 mm and the length less than 50 mm. In one embodiment, the host structure 320 includes an opening 322 therein for access to the interior area 20A of the host structure 320. In one embodiment, a retractable, removable or pivotable member 24 (e.g., a door, window or flap) selectively covers the opening 322. Propulsion systems 30A and 30B may be used to move device 5 within lumen 300. Additionally, propulsion systems 30A and 30B may be used to as orientation control device 31A and 31B. The propulsion systems can generate smaller and or finer movements to maintain the position of the device within the lumen 300 and can be used to change the orientation of the device within the lumen 300, tissue, muscle or fat. Controlling the orientation of the medical device 5 within the lumen 300, tissue, muscle or fat allows the intra-device storage system, imaging system, therapy system, sample and data gathering system, and/or a material dispensing system to be adjacent to a region of interest within the lumen, tissue, muscle, bone marrow or fat.

As shown in FIG. 1B, a first power supply 40A and a second power supply 40B are in communication (e.g., via power supply conductors or transmission lines or channels generally designated by the dashed lines marked 11P) with the first propulsion system 30A and the second propulsion system 30B. While the first power supply 40A and the second power supply 40B are shown and described as being in communication with the first propulsion system 30A and the second propulsion system 30B, the present invention is not limited in this regard as only one power supply or more than two power supplies may be employed and any of the power supplies (e.g., 30A or 30B) may be in communication with one or more propulsion systems (e.g., 40A or 40B).

As shown in FIG. 1B, a control unit 350 is in communication (e.g., via signal transmitting lines, wires or wireless channels, generally designated by dashed lines marked 11S) with the first propulsion system 30A, the second propulsion system 30B, the first power supply 40A and the second power supply 40B. The control unit 350 includes a computer process controller 355 that is configured to control the first propulsion system 30A, the second propulsion system 30B to move the host structure 320, the first propulsion system 30A and the second propulsion system 30B in the lumen 300 so that the host structure 320, the first propulsion system 30A, the second propulsion system 30B and the control unit 350 are self-maneuverable within the lumen 300.

As shown in FIG. 1B, a tracking device 351, a signal transmitter 352 and a signal receiver 353 are in communication with the control unit 350 via signal lines 11S for tracking and guiding the medical device 5 within the lumen 300.

As shown in the exemplary embodiment of FIG. 2, the intra-body controllable medical device 5 may be octopus shaped. The intra-body controllable medical device has a main body 30, and appendages 35. Appendages 35 may be used for propulsion, covering or wrapping the host structure 20, forming a portion of the host structure 20 or to perform a therapeutic or diagnostic task. A control unit, power supply systems, an intra-device storage system, an imaging system, a therapy system, a sample and data gathering system, and a material dispensing system similar to those shown and described with reference to FIG. 1B, may be located within main body 30 and/or appendages 35 of the device or in the interior areas 22 of the host structure 20.

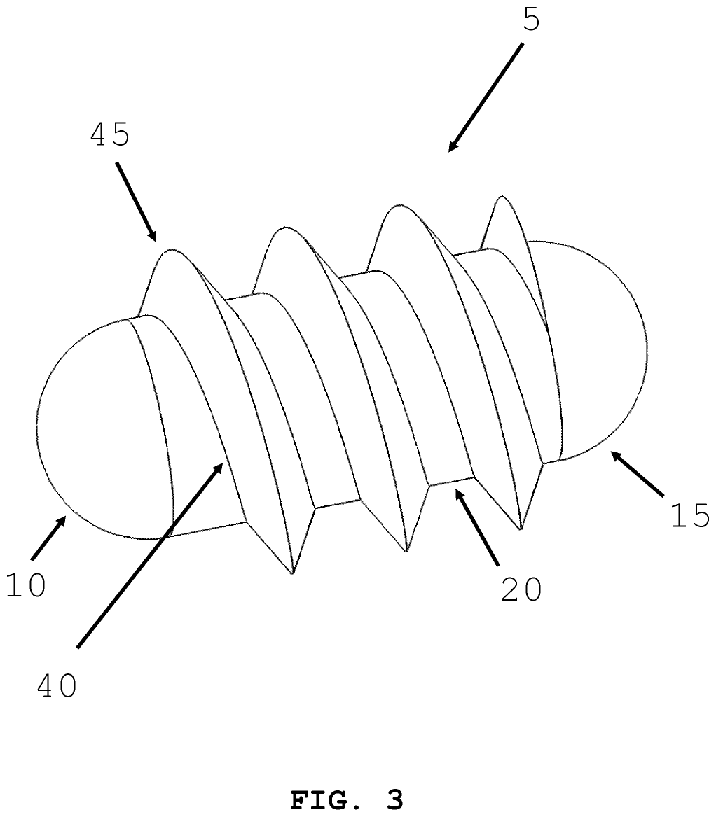

As shown in FIGS. 3-11, the present invention is generally directed to an intra-body controllable medical device and more particularly to propulsion systems for moving the intra-body controllable medical device within a lumen or orifice. The propulsion systems include one or more orientation control devices 31A, 31B per FIG. 1B, for controlling the orientation of the device within the lumen or orifice. The intra-body controllable medical device is sized to travel through lumens and/or orifices tethered and/or untethered. Thus, the intra-body controllable medical device is equipped with one or more propulsions systems, including but not limited to: (1) a sprocket driven track structure in communication with the device; (2) fluid jet stream discharging from the device; (3) an arrangement of inflating and deflating balloons in predetermined positions on and/or around the device; (4) a plurality of articulating tentacles extending from the device; (5) a screw-drive formed on external surfaces of the device and (6) stabilization wings, flippers, anchors, braces, supports and/or clamps, as described herein. The intra-body controllable medical device may also move within the body through peristalsis of the digestive system. In one embodiment, a propulsion system may be used to move device 5 to a region of interest. The device may then exit the body passively through peristalsis or may be withdrawn from the body by a tether.

Referring to FIG. 3, an intra-body controllable medical device with a screw-drive propulsion system is shown. The screw drive propulsion system has a helical thread on the external surface of the device. The helix 40 circumscribes the body 20 of the intra-body controllable medical device. A screw thread 45 is swept around the helix 40. Rotation of the helix 40 around the central axis of body 20 causes the intra-body controllable medical device to advance in the lumen 300 or orifice. Switching the direction of rotation of the helix 40 causes the intra-body controllable medical device to advance in the opposite direction.

In an alternative embodiment and referring to FIG. 4 and FIG. 5, an intra-body controllable device with a sprocket driven track structure in communication with the device is shown. The track 50 may be oriented either along the axis A of the body 20 (FIG. 4), circumferentially around body 20 (see arrow C in FIG. 5) or along one or more portions of the host structure 20 (see the second propulsion system 30B in FIG. 1B). A sprocket (not shown) may be housed within the proximal end 10 and distal end 15 of the device (FIG. 4) or central to the body 20 (FIG. 5). Movement of the track 50 relative to the body 20 of the intra-body controllable medical device 5 generates motion of the medical device.

In an alternative embodiment and referring to FIG. 6, an intra-body controllable medical device with a fluid/gas jet stream discharge propulsion system is shown. The jet stream 55 of mater (e.g., gas, liquid, gel, or particles) may be released from intra-body controllable medical device 5 through a nozzle or orifice 60. The orifice 60 may be located on the distal end 10 and/or the proximal end 15 of the device. The jet stream 55 matter may be stored within body 20 of the device. Alternatively, the matter may be harvested from the body (e.g. gastric juice). Alternatively, the fluid may be harvested from the body (e.g. gastric juice) and reacted with a compound stored within device 20 (e.g. sodium bicarbonate) to create a gas (e.g. carbon dioxide gas) which can be released as a fluid/gas jet stream 55 under pressure through the orifice 60 to create propulsion. Additionally, a propeller and or turbine 61 may be located within nozzle or orifice 60. The jet stream of matter 55 may turn the turbine to generate thrust. Additionally, fluid/gas jet stream discharge propulsion system may be used as an orientation control system 31A and 31B.

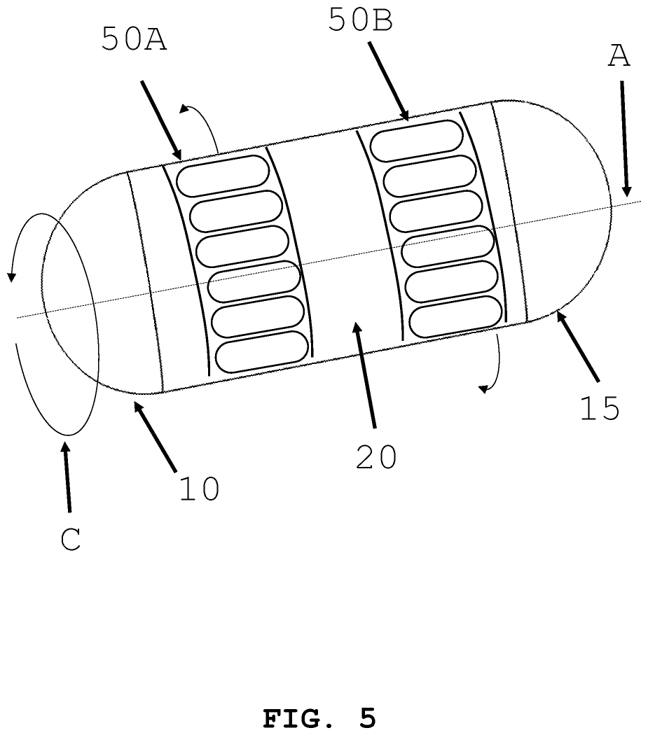

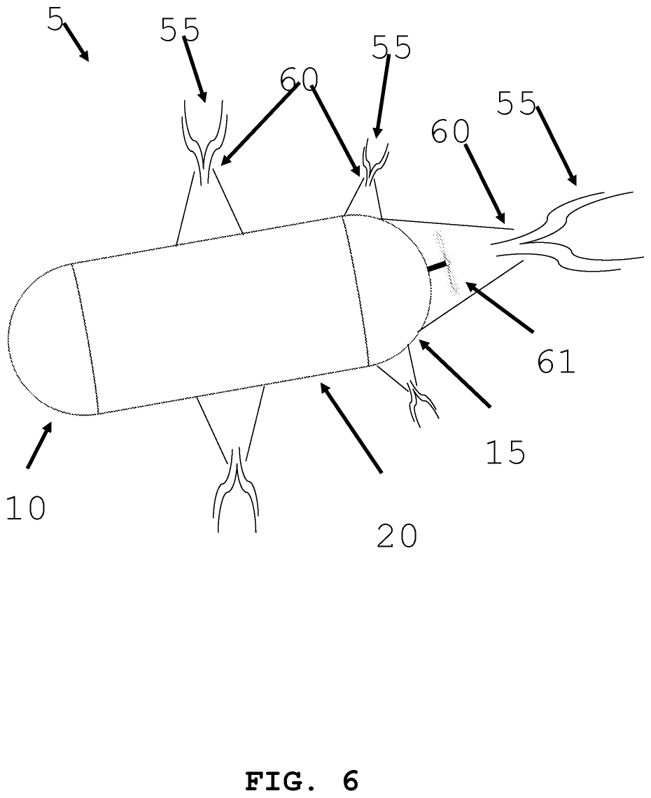

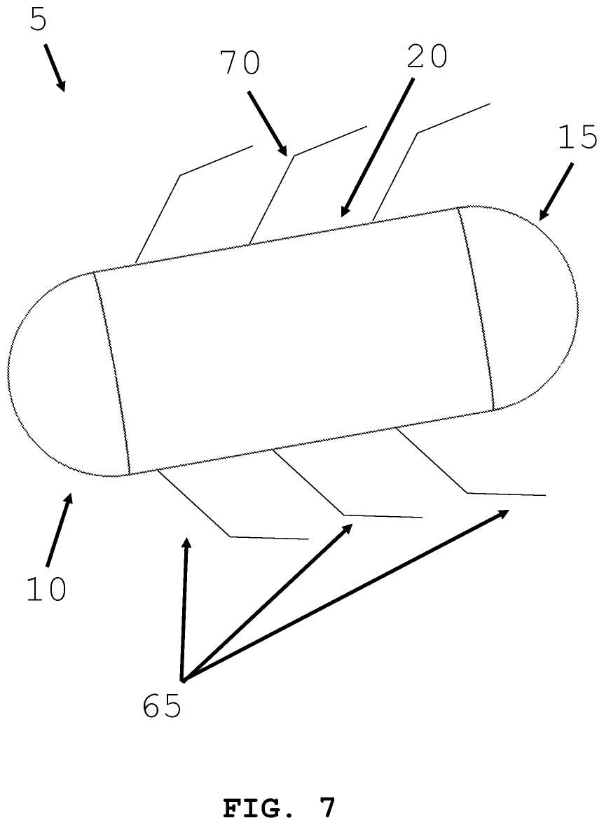

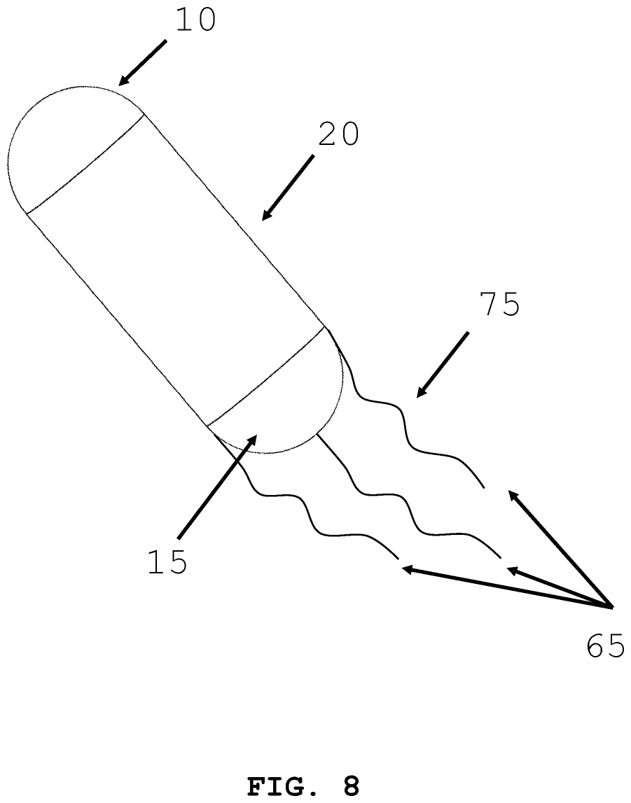

In an alternative embodiment and referring to FIG. 7 and FIG. 8, an intra-body controllable medical device 5 with a plurality of articulating tentacles extending from the body is shown. A plurality of tentacles 65 may be disposed along the length of the body 20 of the device (FIG. 7); alternatively, the tentacles 65 may be located on the distal end 10 or proximal end 15 of the device (FIG. 8). The tentacles 65 may be linear. The tentacles 65 may be linear with hinged regions 70 or may be able to articulate throughout their length 75. Motion of the tentacles 65 generates propulsion of the intra-body controllable medical device.

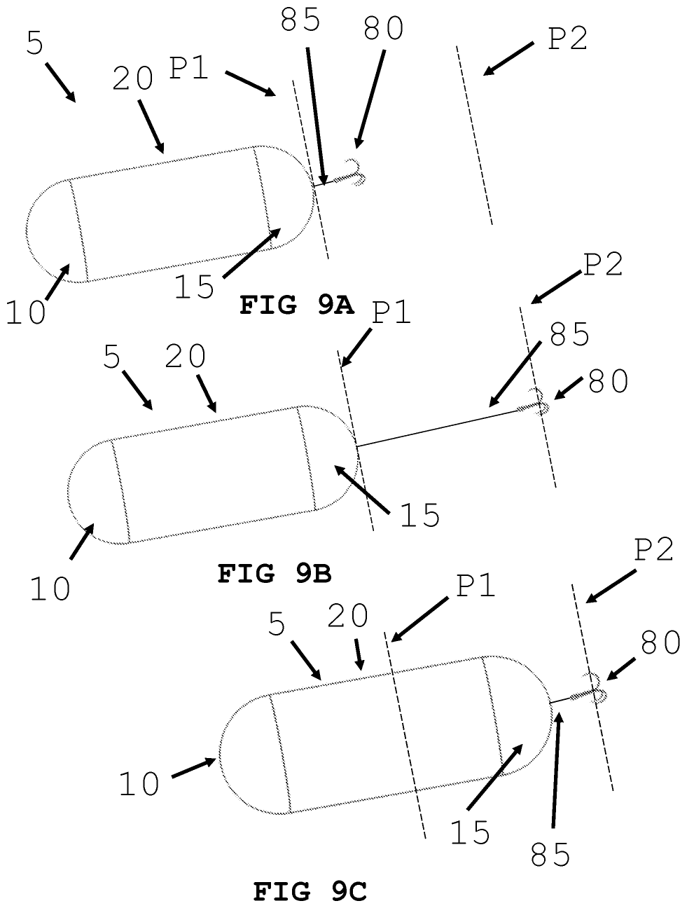

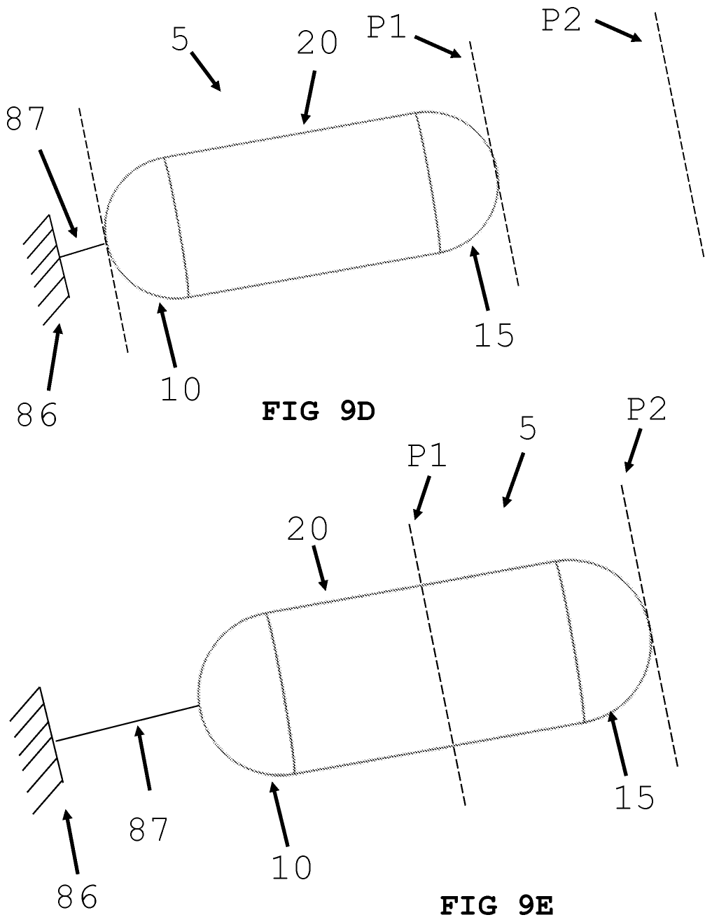

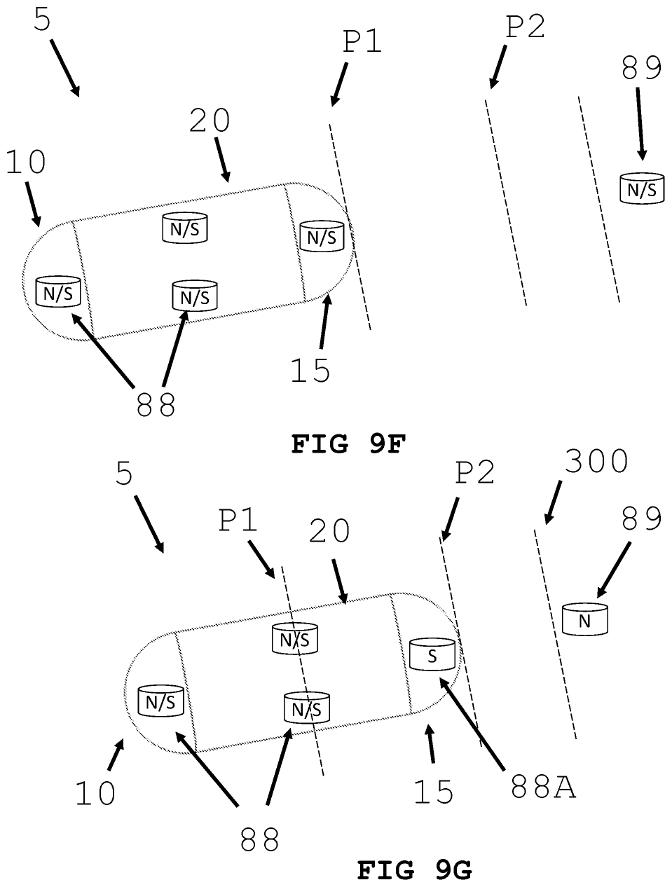

In an alternative embodiment and referring to FIGS. 9A-9G, an intra-body controllable medical device 5 with a push or pull propulsion system is shown. As shown in FIGS. 9A, 9B, and 9C, a retractable anchor-based propulsion system is shown. An anchor 80 can be any kind of anchor known in the art. As shown in FIG. 9A, the proximal end 15 is at position P1 and the anchor 80 is in the retracted position. As shown in FIG. 9B, the anchor 80 is deployed via an extended tether 85 and attaches to tissue at position P2. The anchor 80 is connected to the intra-body controllable medical device 5 by a tether 85. Propulsion is generated by retracting the tether 85 (FIG. 9C), thereby pulling the medical devices to the position P2.

In an alternative embodiment and referring to FIGS. 9D and 9E a push propulsions system is shown. As shown in FIG. 9D the proximal end 15 is at position P1 and push rod 87 is in the retracted position. The end of push rod 87 may be adjacent to a fixed structure 86. Fixed structure 86 may be lumen 300, a probe, or a scope. Propulsion is generated by advancing push rod 87 (FIG. 9E) thereby pushing the medical device to the position P2.

In an alternative embodiment and referring to FIG. 9F and FIG. 9G a push and or pull propulsion system is shown. As shown in FIG. 9F, push and or pull propulsion system utilizes magnets or magnetic fields to move device 5. Magnets may be permanent or electromagnetic. Magnets 88 are located within device 5. Additionally, there may be one or more magnets 89 located outside of lumen 300. Magnets 88 and 89 are configured to have either a north pole or a south pole. Magnets 89 may be located outside of the organism. Proximal end 15 is located at position P1. Propulsion is generated by creating an attraction force between magnet 89 and magnets 88 (FIG. 9G). An attractive force is generated between magnet 88A's south pole and magnet 89's north pole. This attractive force moves the medical device to position P2. Alternatively, magnet 88A's south pole (or north pole) may be aligned with magnet 89's south poll (or north pole), a repulsive force can be generated and used to push medical device 5.

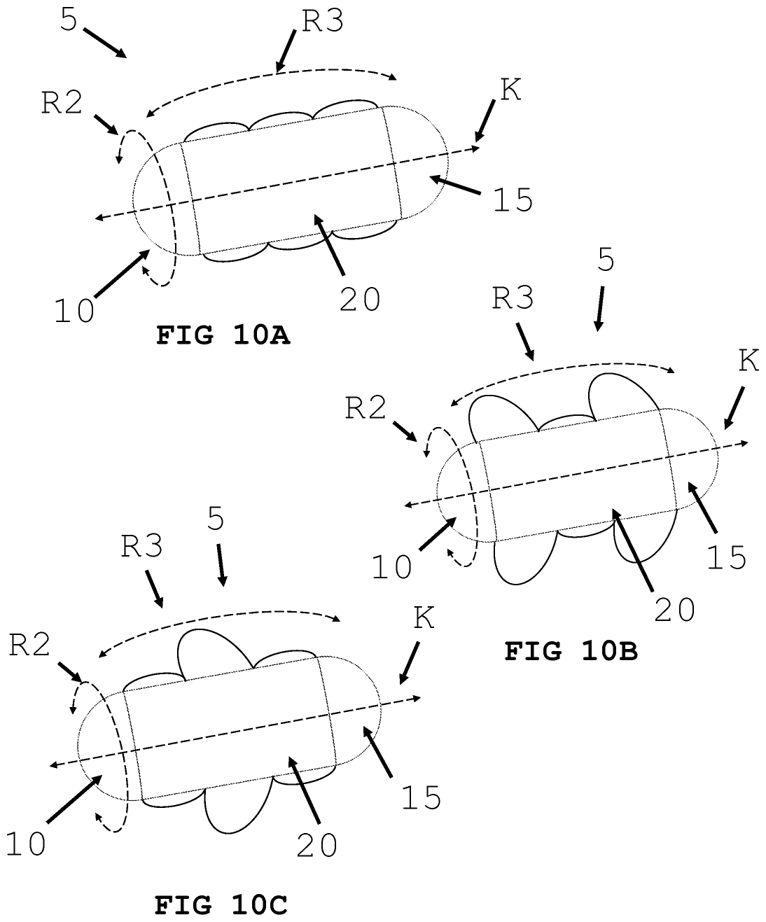

In an alternative embodiment and referring to FIG. 10, an intra-body controllable medical device 5 with an arrangement of inflating and deflating balloons 90 in predetermined positions in the direction of the arrows R and orientations (e.g., rotational or angular movement as indicated by the arrows R2 and R3) on and/or around the device is shown. The balloon 90 may be made of an elastomeric material that can be expanded under pressure yet return to its original configuration when the pressure is released. The balloon 90 may be filled with a fluid and/or a gas. When the balloon 90 is filled, the balloon increases in volume and changes shape. As an example, the balloon 90 may change to shape conformation 95 when filled with a fluid and/or gas. The fluid and/or gas may be stored within the body 20 of the medical device 5. Alternatively, the fluid may be harvested from the body (e.g. gastric juice). Alternatively, a fluid may be harvested from the body (e.g. gastric juice) and reacted with a compound stored within the device (e.g. sodium bicarbonate) to create a gas (e.g. carbon dioxide). This gas can then be used to fill and expand the balloon 90. A controller can be located within the device to direct the fluid and/or gas flow to different balloons. The rhythmic expansion and contraction of balloons can create propulsion.

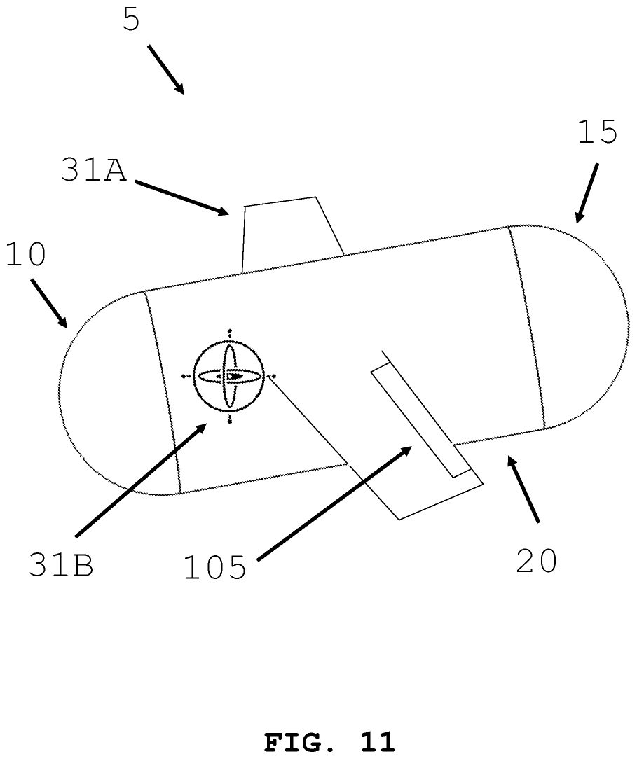

In an alternative embodiment and referring to FIGS. 1B and 11, an intra-body controllable medical device 5 may be equipped with an orientation control device (e.g. stabilization wing 31A, 31B). The orientation control device (e.g. stabilization wing 31A, 31B) is compatible with any of the propulsion systems disclosed herein. The orientation control device (e.g. stabilization wing 31A, 31B) can help guide the movement of the medical device 5 within the lumen. The orientation control device (e.g. stabilization wing 31A, 31B) may further have a flap 105 to further provide stabilization and guidance. Orientation control device may also be a gyroscope 31B. Gyroscope 31B may be used to provide stability or maintain a reference direction.

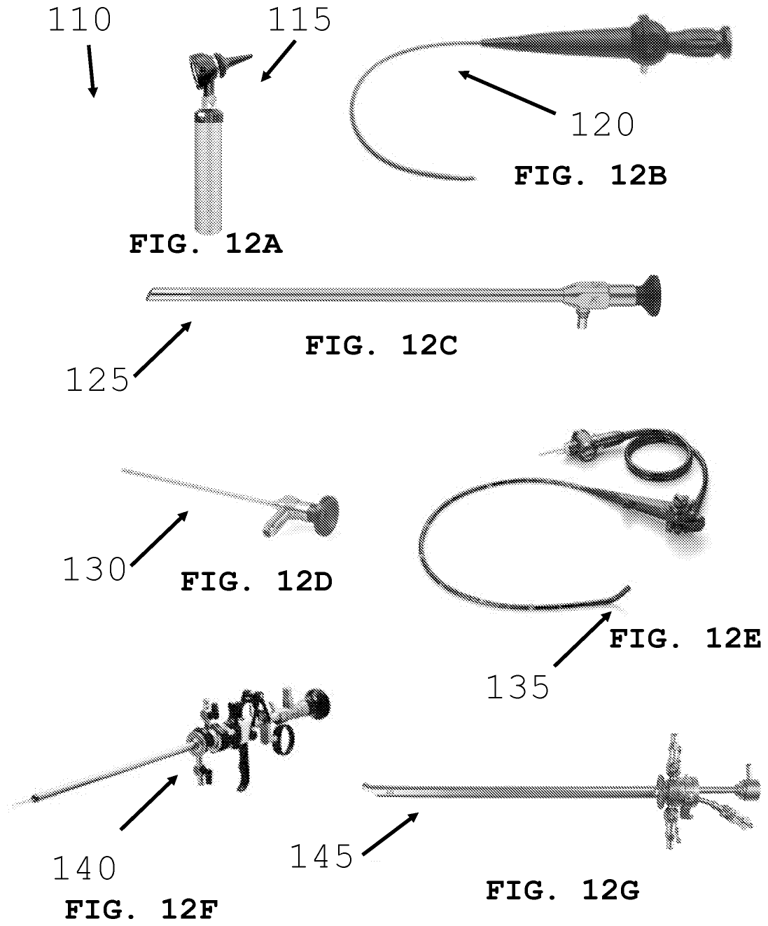

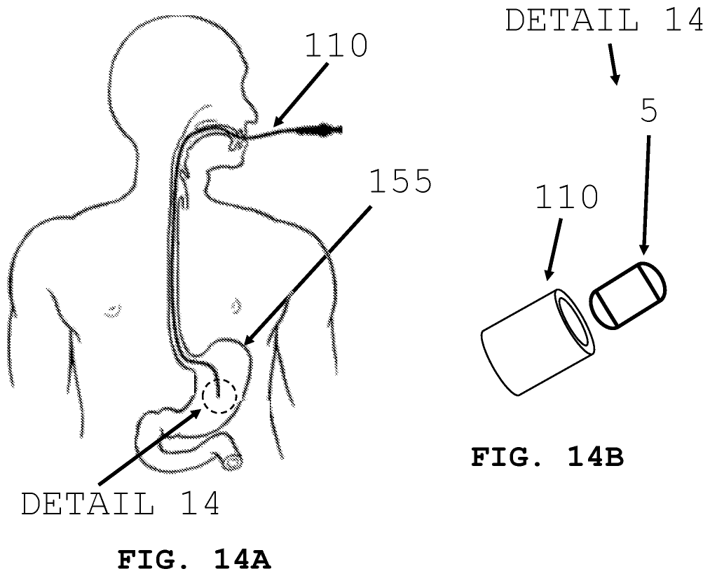

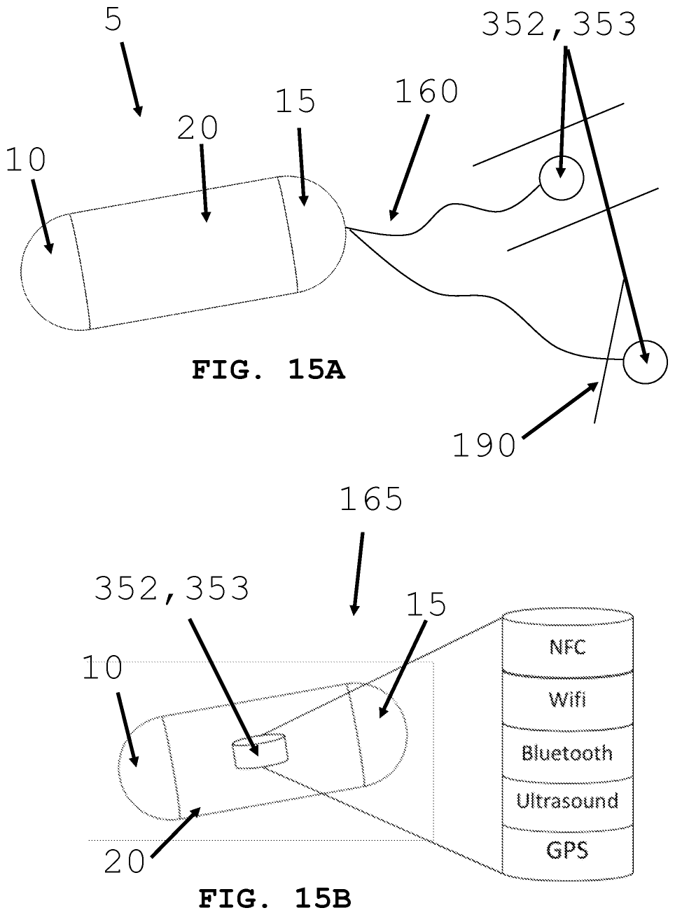

As shown in FIGS. 12A-G through FIG. 14, the present invention is generally directed to an intra-body controllable medical device 5 and more particularly to deployment devices and methods for deploying an intra-body medical device into the lumen 100. In particular, scopes for medical applications having rigid shafts or flexible conduits are configured with one or more device storage compartments, channels, actuation devices, tethers and discharge ports for deployment from one or more portions of the probe portion of the scope while positioned in a lumen. Referring to FIG. 12, the deployment device is configured to be integrated with various medical scopes 110 including but not limited to an ENT otoscope 115, a naso-pharyngoscope 120, a laparoscope 125, a sinuscope 130, a colposcope 135, a resectoscope 145 and a cystoscope 150. Furthermore, medical device 5 may be deployed through a tube instead of a scope. Additionally, medical device 5 may be deployed via a catheter into a blood vessel or may be surgically placed (e.g. after heart surgery). Medical device 5 may be deployed though an appropriately sized needle (e.g. to gain access bone marrow) and may also be deployed generally to any area within the body (e.g. muscle, fat, and tissue) or on the skin. Medical device 5 may be deployed on the skin at the site of a wound and provide therapy (e.g. discharge clotting material like zeolite or antibacterial medication).

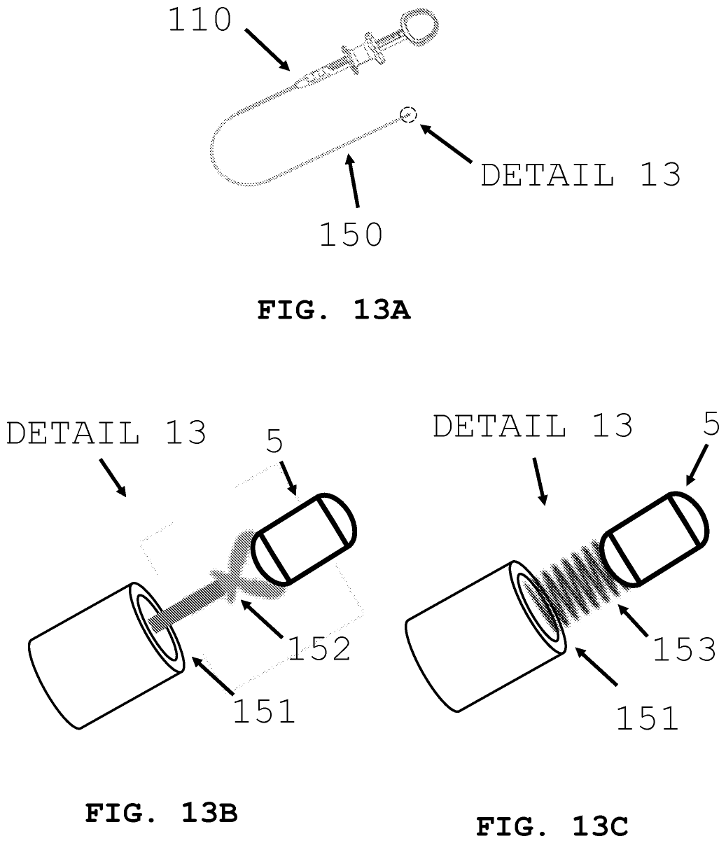

As shown in FIG. 13, the intra-body controllable medical device 5 can be deployed through the working channel 150 of the endoscope 100. The end of the working channel 150 may have a docking station 151 (FIG. 13B and FIG. 13C.) Docking station 151 may utilize a claw 152 (FIG. 13B) or a spring 153 (FIG. 13C) to hold and deploy medical device 5. Furthermore, and referring to FIG. 14A and FIG. 14B this method for deployment of the intra-body controllable medical device in the lumen 100 further includes the use of a scope 110 to deliver the device directly to the stomach 155. Alternatively, the scope 110 may be used to deliver the device directly to various organs, for example, the bladder. The method for deployment of the intra-body medical device in a lumen further includes digestion through the oral cavity, inhalation of one or more nano-sized versions of such devices for introduction to the respiratory system of a human, including the nose, pharynx, larynx, trachea, bronchi and lungs.

As shown in FIG. 15A and FIG. 15B, the present invention is generally directed to an intra-body controllable medical device 5 and more particularly to control and communications systems and methods for controlling and communicating with the intra-body controllable medical device in a lumen. In particular, the control and communications systems are configured to identify and track the location and orientation of the device relative to predetermined locations in the lumen and to control the device propulsion and orientation systems to guide the device to, from and around the predetermined position.

As shown in FIGS. 1B and 15A and B, the control unit 50 includes hard wired 160 (FIG. 15A) and/or wireless 165 (FIG. 15B) communication devices (e.g., transmitters 352 and receivers 353) linking an external command and monitor center with a computerized process controller 55 (FIG. 1B) in the medical device 10 which is in communication with and controls the operation of the propulsion and orientation systems based upon real time position information of the device in the body. The control unit 50 includes a software algorithm on a computer readable medium that is operable with the computerized process controller to effectuate the identification, tracking and control of the intra-body controllable medical device within the lumen.

The control unit 350 includes tracking devices 351, transmitters 352 and receivers 353, see FIG. 1B and FIG. 15 including GPS, radiation emitting sources/radiation monitoring devices, ultra sound devices, near field communication devices, Wi-Fi devices, and Bluetooth devices, that are configured to determine the position of the intra-body controllable medical device in the lumen, similar to those shown and described with reference to element numbers 315, 352 and 353 in FIG. 1B.

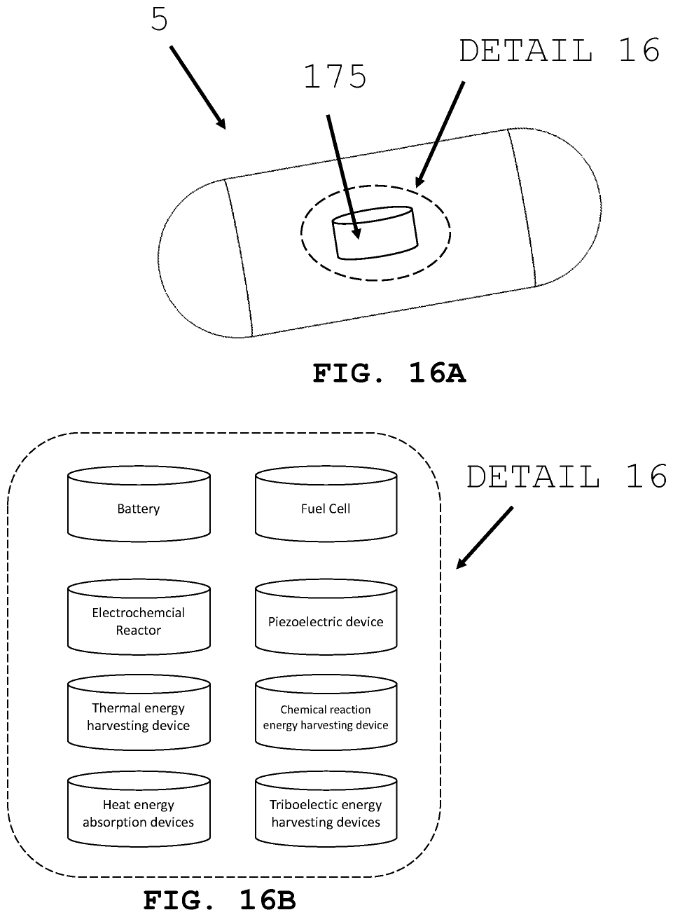

As shown in FIG. 16A and FIG. 16B, the present invention is generally directed to power supply systems 175 for an intra-body medical device and more particularly to miniaturized (e.g., computer chips having integrated circuits and positioned on integrated circuit boards [The intra-body controllable medical device may be sized according to the anatomy that it will need to navigate and the method used to deliver it. As an example, overall dimensions for an intra-body controllable device operating within the gastrointestinal track may have a diameter of about 25 mm and a length of about 75 mm. More preferably, the device may have a diameter of about 15 mm and a length of about 50 mm. Most preferably, the diameter may be less than about 15 mm and a length of less than about 50 mm. Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15 mm and the length less than 50 mm. Control system, power supply system, intra-device storage system, imaging system, therapy system, sample and data gathering system, and material dispensing systems are sized to fit within these dimensional guidelines.]) power supplies and storage devices that provide power for propulsion, control and operation of subcomponents within and around the intra-body controllable medical device and ancillary devices connectable to the intra-body controllable medical device. In particular, the miniaturized power supplies include batteries, fuel cells, electrochemical reactors, piezoelectric devices, energy harvesting devices that obtain thermal and/or chemical reaction energy from the fluids in and tissue of the lumen and adjacent organs, thermal reactors heat absorption energy conversion devices and triboelectric energy harvesting devices. Batteries may include any of the kind known in the art including, but not limited to, alkaline batteries, atomic batteries, lead-acid batteries, lithium ion batteries, magnesium-ion batteries, nickel-cadmium batteries, nickel metal hydride batteries and rechargeable alkaline batteries. Electrochemical reactors may store the chemical required to create electricity within the device. Alternatively, electrochemical reactors may use fluids found within the body to react with chemicals stored within or on the device to create electricity. Piezeoelectric devices may create electricity by harvesting either the body's own motion (e.g. peristalsis) or the motion of the device as it moves within the lumen. Heat absorption devices may harvest energy from the body's temperature to create electricity. Triboelectric energy harvesting devices generate electricity as the body of the device comes into frictional contact with the lumen of the body it is passing within. Additionally, energy may be stored by the device using capacitors, thermal medium, batteries and mechanical expansion devices (e.g., springs and balloons).

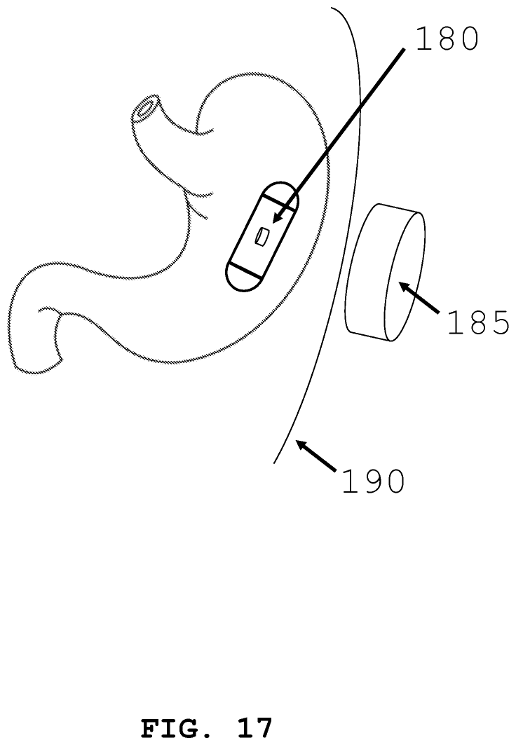

Additionally, as seen in FIG. 17, the intra-body controllable medical device 5 can be directly powered by induction energy transfer from the outside of the body 190 or inside of the body 190. An induction energy receiver 180 can be located within device 5. An induction energy transmitter 185 can be located outside body 190. Alternatively, the device may function on another internal energy storage device and be recharged by induction, charging when sufficient stored electricity has been consumed.

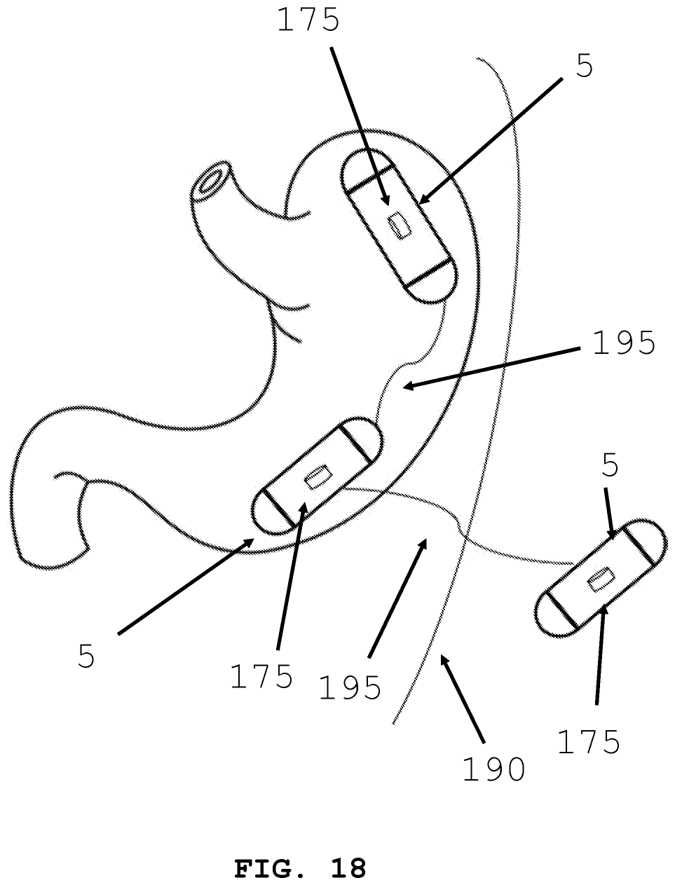

Alternatively, as seen in FIG. 18, one intra-body controllable medical device 5 can be tethered to a second intra-body controllable medical device 5. Tether 195 can transfer electricity from power source 175 in a first device to a second power source 175 of the second device. The second intra-body controllable medical device 5 may be located outside the body 190. The two devices may be permanently tethered together, or they may tether when the transfer of electricity is required.

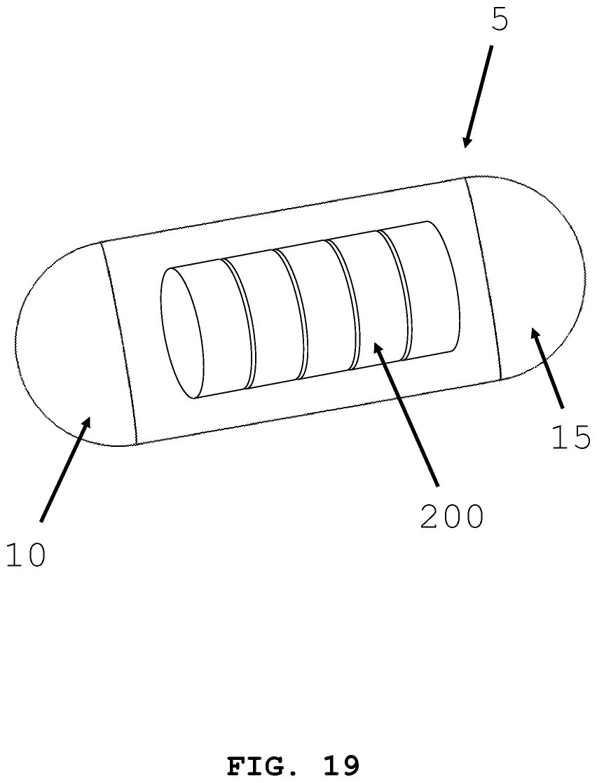

As shown in FIG. 19, the present invention is generally directed to an intra-body medical device having intra-device storage systems 200 therein and more particularly to miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables. The storage compartments are configured with walls, internal and external support structures, inlets, outlets, sensors (e.g., temperature, pressure and chemistry sensors), valves, pumps and ingress/egress apertures. The intra-device storage system 200 may be used to hold nerve blocking and stimulating drugs and devices, may hold devices for cleaning plaque from artery walls or may hold and deploy intestinal restrictive bands.

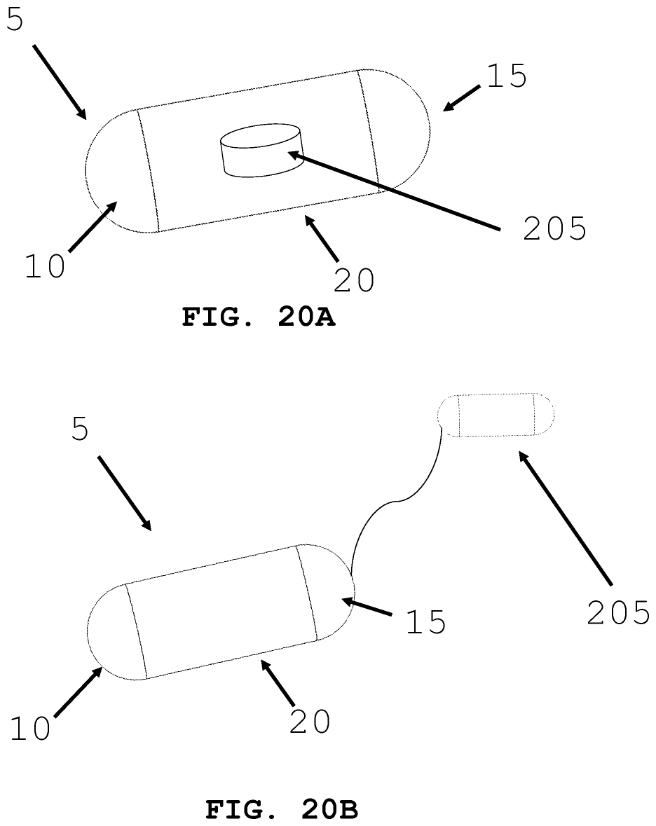

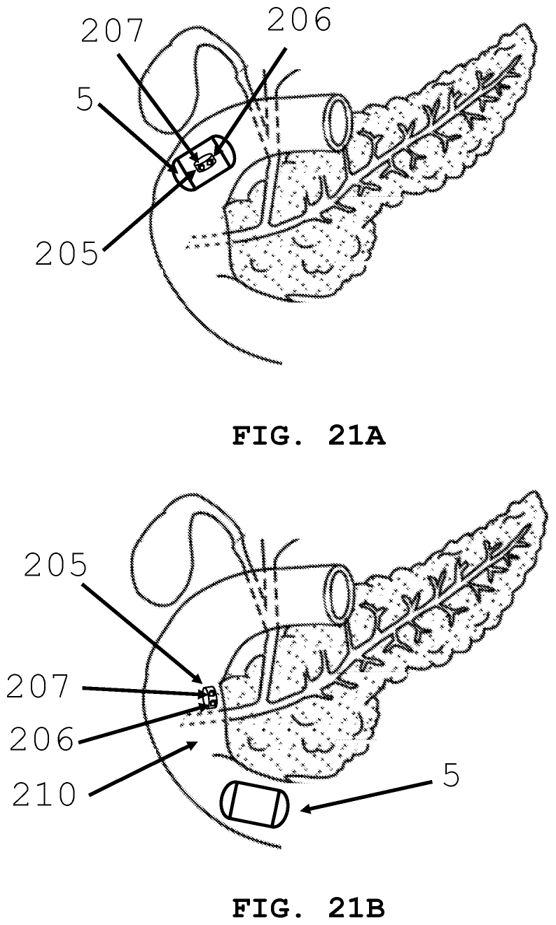

As shown in FIG. 20A, FIG. 20B and FIG. 21 the present invention is generally directed to an intra-body controllable medical device having one or more imaging systems 205 within (FIG. 20A) or remote (FIG. 20B) to the intra-body controllable medical device. The imaging systems include X-ray radiography, magnetic resonance imaging, medical ultrasonography or ultrasound, confocal microscopy, elastography, optical-coherence tomography, tactile imaging, thermography and medical digital photography. In one embodiment, the imaging systems 205 are configured to travel through the lumen 300 in the intra-body controllable medical device (FIGS. 20A and 20B). In an alternative embodiment and referring to FIG. 21A and FIG. 21B, the imaging systems 205 are further configured to be discharged from the intra-body controllable medical device while in the lumen and deposited in a predetermined location in the lumen for ongoing monitoring. As an example, and referring to FIG. 21A, the medical device 5 may be equipped with imaging system 205. The medical device 5 may travel through the small intestine and deposit imaging system 205 adjacent to the Ampula of Vata 210 (FIG. 21B). The medical device 5 may then continue to travel through the small intestine without the imaging system. The imaging systems 205 may be configured with a storage medium 206 to store images. The imaging systems 205 may further be configured with a transmitting device 207 to transmit real time images to one or more receivers located in other positions in the lumen and those located in other locations and organs in the body (e.g., a human body) and outside of the body.

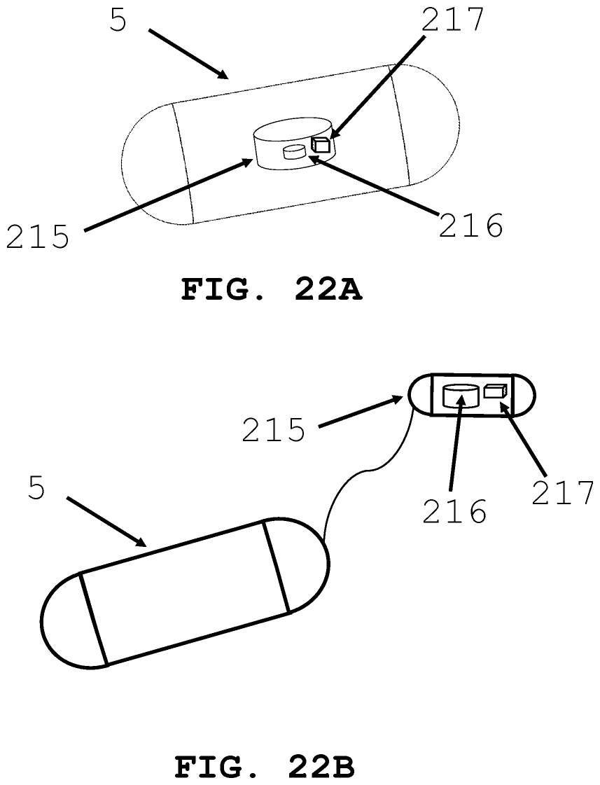

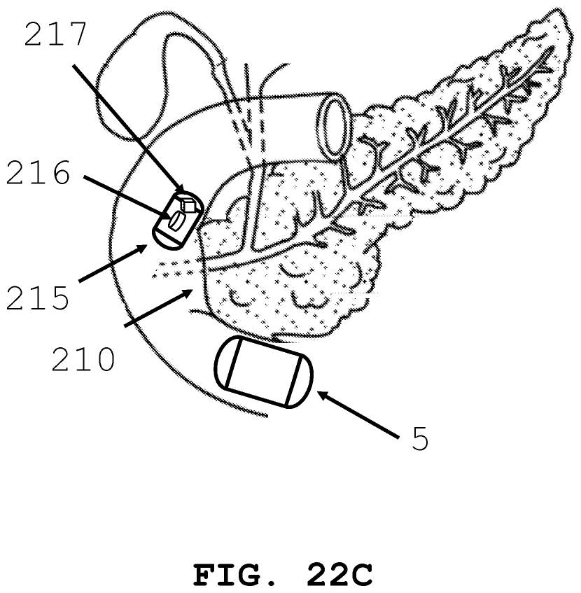

As shown in FIG. 22A, FIG. 22B, and FIG. 22C, the present invention is generally directed to an intra-body controllable medical device having one or more therapy delivery systems 215 within (FIG. 22A) or remote (FIG. 22B) to the intra-body controllable medical device. The therapy delivery systems 215 include optical-coherence tomography (OCT) guided laser instruments, radiation discharging sources, chemotherapy deploying devices, pharmaceutical and drug deploying devices, ablation devices and photodynamic therapy devices. The therapy delivery systems 215 are configured to travel through the lumen in the intra-body controllable medical device and provide therapy. The therapy delivery systems 215 may further be configured to be discharged from the device while in the lumen 100 and deposited in a predetermined location in the lumen 100 for ongoing therapy delivery (FIG. 22C). The therapy delivery systems 215 may be configured with a storage medium 216 to record time, duration and application location of the therapy. The imaging systems 205 and therapy device systems 215 may be further configured with a transmitting device 217 to transmit real time images to one or more receivers located in other positions in the lumen and those located in other locations and organs in the body (e.g., a human body) and outside of the body. The medical device 5 of FIG. 22A and FIG. 22B may travel through the small intestine and deposit therapy delivery system 215 adjacent to the Ampula of Vata 210 (FIG. 22C). The medical device 5 may then continue to travel through the small intestine without the imaging system.

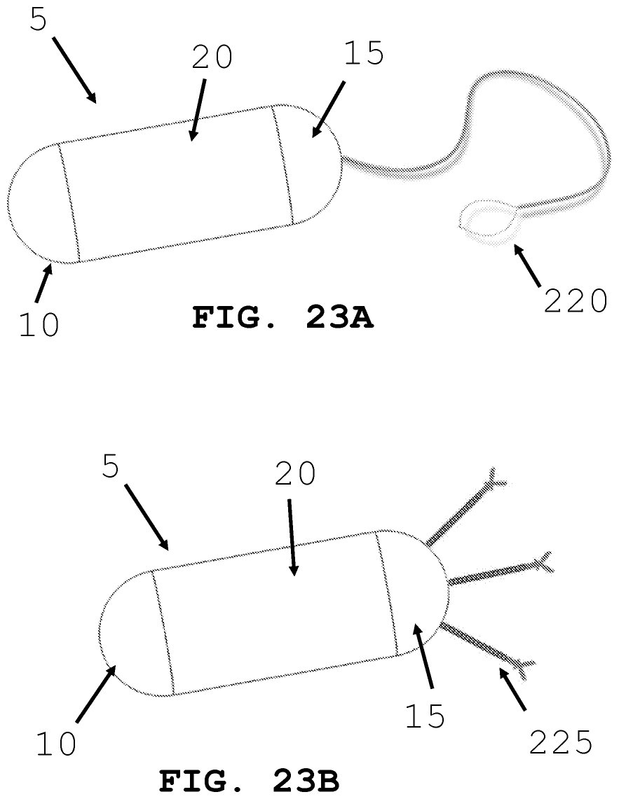

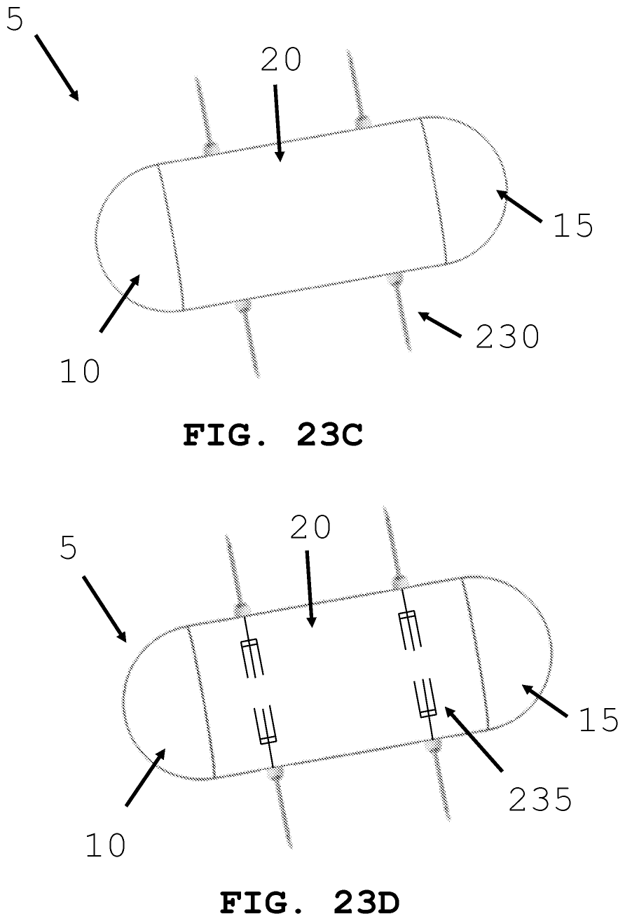

As shown in FIG. 23, the present invention is generally directed to an intra-body controllable medical device having one or more sample and data gathering systems. The sample gathering systems are configured to obtain tissue biopsies and blood, bone, cells, bone marrow, blood, urine, DNA and fecal samples. The sample gathering devices may include any known in the art including snares 220, forceps 225, and needles 230. The data gathering devices may include pH probes, accelerometers, pressure transducers, thermometers, and dimensional measurement systems. The sample and data gathering systems are configured to perform localized testing such as complete blood counts, bone density measurements, acidity testing and turbidity testing. The sample and data gathering systems are configured to take, record and transmit dimensional, angular, velocity and volumetric measurements. The intra-body controllable medical devices contain miniaturized devices for performing the tests and obtaining the data, including miniaturized needle aspiration devices 230, and suction devices 235. The dimensional, angular, velocity and volumetric measurements are acquired by miniaturized devices deployed from the intra-body controllable medical device including ultrasound systems and laser imaging.

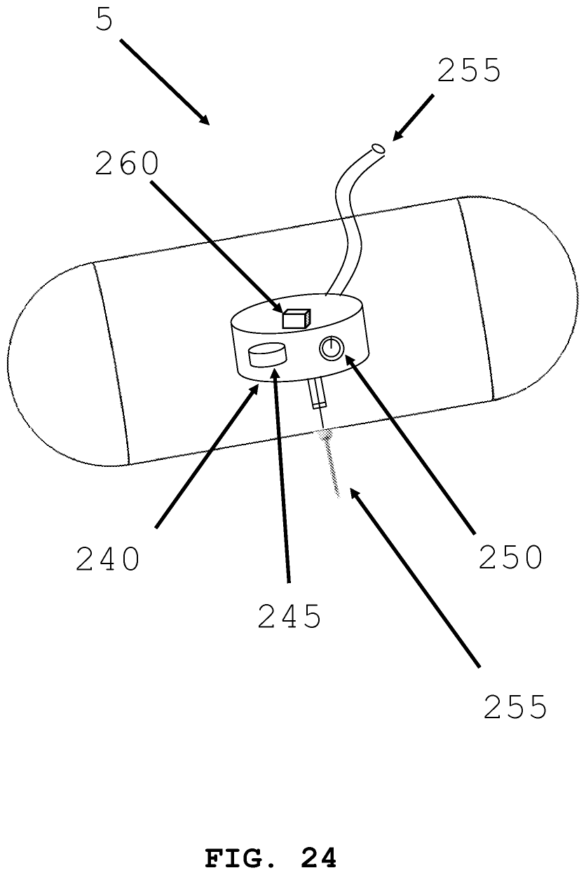

As shown in FIG. 24, the present invention is generally directed to an intra-body controllable medical device having one or more material dispensing systems. The material dispensing systems 240 are equipped with storage compartments 245 for storing and dispensing payloads including medication, liquids, powders, chemically reactive agents and radiation emitting sources and recording and tracking the location of the payloads before and after the dispensing operation. The material dispensing systems include actuators, pumps, compressors, nozzles, flow control devices 250 including valves and orifices, injection and piercing devices 255 and dose measuring and recording devices 260.

The present invention is generally directed to materials for manufacture of an intra-body controllable medical devices, and in particular to materials for such devices that are clinically inert, sterilizable, elastomeric (e.g., contractible and expandable), chemically reactive, chemically inert, dissolvable, collapsible and have physical and chemical properties to withstand exposure to bodily fluids for precise predetermined periods of time. Such materials include polymers, metallic alloys, shape memory polymers, shape memory metal alloys, shape memory ceramics, composites, silicones, thermoplastic polyurethane-based materials, excipients, zeolite adsorbents and styrene-butadiene rubbers (SBR). Materials may further include biodegradable materials such as paper, starches, biodegradable material such as gelatin or collagen.

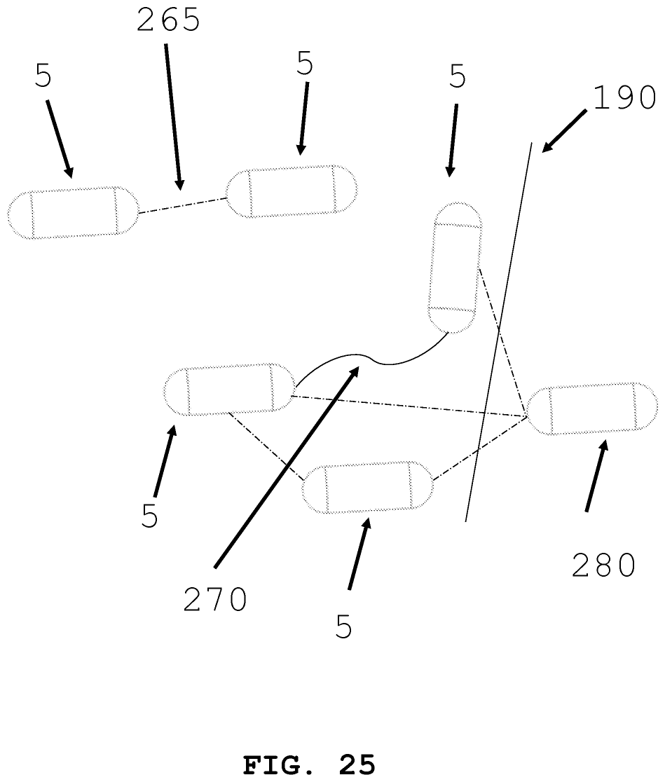

As shown in FIG. 25, the present invention is generally directed to an interactive group of intra-body controllable medical devices. The interactive group of devices includes two or more devices 5 that are in communication with one another and/or an external computer-based control system. The two or more intra-body controllable medical devices are configured to cooperate with one another to distribute components such as power supplies, medical devices, storage compartments and auxiliary devices among the intra-body controllable medical devices so that the intra-body controllable medical devices operate together as a group to accomplish the intended functional operations and to enable the use of smaller sized individual intra-body controllable medical devices than those that would otherwise not fit into the lumen. The interactive group of intra-body controllable medical devices is configured to operate collectively as a swarm of a plurality of intra-body controllable medical devices that if deployed individually would not be as effective in undertaking the intended medical procedure or other functional operation. The interactive group of intra-body controllable medical devices includes tethering 270 or towing devices (e.g., winches) between intra-body controllable medical devices to assist in propulsion of the intra-body controllable medical devices through the lumens. Additionally, the intra-body medical devices may communicate wirelessly 265 between devices. Intra-body medical devices may communicate with a receiver or controller 280 located outside the body 190. Intra-body medical device 5 may operate like a drone, communicating and being controlled by an operator in the same room or in a different location from the patient. Furthermore, when contemplating a swarm of devices, two or more intra-body controllable medical devices 5 may be deployed. A first intra-body medical device 5 may leave the swarm group and navigate to a region of interest. This device may perform a first task and communicate back to the other devices in the swarm and direct a second device 5 to navigate to the first device 5. Second device 5 may be selected from a number of devices in the swarm because of its particular capabilities (e.g., second device 5 may have an additional battery, an imaging system, a therapy system, a sample and data gathering system, and/or a material dispensing system). Second device 5 may transfer capabilities to first device 5 or second device 5 may perform a task related to its specific capabilities. This serial communication and deployment of devices from the swarm may continue until the desired procedure is completed.

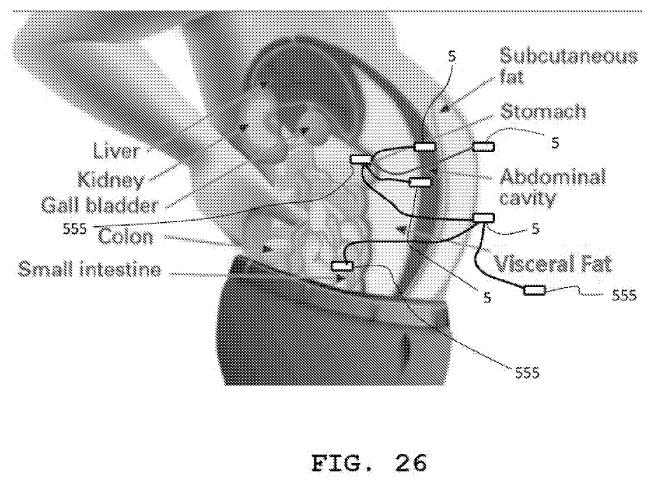

As shown in FIG. 26, intra-body medical device 5 or an interactive group thereof may be used to deliver cold therapy within the patient. A plurality of medical devices (5) may be in communication with one or more repositories (555). The repositories 555 include heat sinks, heat exchangers, chemical reactors and/or storage vessels. The plurality of medical devices has a cooling system and/or a material discharge system disposed therein or thereon. The repositories 555 are positioned intra body (i.e., inside the human body) and/or outside the human body. The intra-body medical devices 5 are shown connected to each other and the repositories 555 by a network of conduits (e.g., tubes, cannulas, capillaries, heat conducting materials and ducts).

The present invention is directed to configurations for intra-body controllable medical devices and in particular to disposable, disintegrable and selectively collapsible intra-body controllable medical device s and materials and structures thereof. The intra-body controllable medical devices are manufactured of a material such as an elastomer (e.g., nitrile) that can expand and contract, for example, by inflating and deflating them. The intra-body controllable medical devices are manufactured from a biodegradable, disintegrable or dissolvable material, including paper, starches, biodegradable material such as gelatin or collagen and/or synthetic natural polymers. The collapsible intra-body controllable medical devices are configured to be flattened, extruded, stretched or disassembled in the lumen. Thus, the intra-body controllable medical devices are disposed of in the lumen or via discharge therefrom without the need to recover the intra-body controllable medical devices for analysis, inspection or future use.

The present invention is directed to methods for using intra-body controllable medical devices in the medical field and in particular for use in administering medications and therapy, deploying medical devices, imaging and surgery. The methods for using intra-body controllable medical devices includes applications in the gastro/intestinal tract (e.g. colonoscopy), urology applications, in the lungs, bladder, nasal and reproductive systems, in performing Transurethral Resection of Bladder Tumors (TURBT), Transurethral Resection of the Prostate (TURP) and transrectal prostate ultrasound, biopsy, and radiation treatment. The methods for using intra-body controllable medical devices include use in procedural environments, operatory/surgical procedures, ambulatory/out-patient procedures and unobtrusive normal routine living.

Although the present invention has been disclosed and described with reference to certain embodiments thereof, it should be noted that other variations and modifications may be made, and it is intended that the following claims cover the variations and modifications within the true scope of the invention.

1. A medical device for intra-body conveyance, the medical device comprising: a host structure defining an interior area;
at least one propulsion system linked to the host structure; the host structure and the at least one propulsion system being configurable into a peripheral boundary of a size adapted to fit in a lumen of a living organism;
at least one power supply in communication with the at least one propulsion system;
a control unit in communication with the at least one propulsion system and the power supply, the control unit having a computer process controller configured to control the at least one propulsion system to move the host structure and the at least one propulsion system in the lumen so that the host structure and the at least one propulsion system are self-maneuverable within the lumen.

2. The medical device of claim 1, wherein the propulsion system comprises at least one of:
a sprocket driven track structure in communication with the host structure;
a fluid jet stream discharging from the host structure;
a plurality of articulating tentacles extending from the host structure;
a screw-drive formed on external surfaces of the host structure;
at least one of a pull device and a push device in communication with the host structure; and
an arrangement of inflating and deflating balloons, the balloons being at least one of: in predetermined positions on the host structure, and
in predetermined positions around the host structure.

3. The medical device of claim 1, wherein the at least one propulsion system comprises an orientation control device configured for orientation control of the medical device within the lumen.

4. The medical device of claim 1, further comprising at least one of a tracking device, a signal transmitter and a signal receiver in communication with the control unit for tracking and guiding the medical device within the lumen.

5. The medical device of claim 1, wherein the host structure comprises at least one storage system comprising miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables.

6. The medical device of claim 1, wherein the host structure comprises at least one of a clinically inert material, a sterilizable material, an elastomeric material, a chemically reactive material, a chemically inert material, a disintegrable material, a dissolvable material, a collapsible material and a material having physical and chemical properties to withstand exposure to bodily fluids for predetermined periods of time.

7. The medical device of claim 1, wherein the host structure comprises at least one imaging system, the at least one imaging system being selected from the group consisting of X-ray radiography, magnetic resonance imaging, medical ultrasonography or ultrasound, confocal microscopy, elastography, optical-coherence tomography, tactile imaging, thermography and medical digital photography.

8. The medical device of claim 1, wherein the host structure comprises at least one therapy delivery system, the at least one therapy delivery system selected from the group consisting of optical-coherence tomography (OCT) guided laser instruments, radiation discharging sources, chemotherapy deploying devices, pharmaceutical and drug deploying devices, and photodynamic therapy devices.

9. The medical device of claim 1, wherein the host structure comprises at least one of a sample gathering system and a data gathering system.

10. The medical device of claim 1, wherein the host structure comprises at least one material dispensing system equipped with at least one storage compartment configured for at least one of storing and dispensing payloads, the payloads comprising at least one of medication, liquids, powders, chemically reactive agents and radiation emitting sources.

11. An interactive group of at least two of the medical devices of claim 1, wherein the interactive group of the at least two medical devices are in communication with at least one of an external computer-based control system and each other and are configured to cooperate with each another to perform at least one predetermined mission.

12. A method for using the medical device of claim 1, the method being directed to at least one of administering medications, administering therapy, deploying medical devices, imaging and surgery.

13. A method for using the medical device of claim 1, the method being directed to at least one of use in a gastro/intestinal tract, use in urology applications, use in a lung, use in a bladder, use in a nasal system, use in a reproductive system, use in performing Transurethral Resection of Bladder Tumors (TURBT), use in Transurethral Resection of the Prostate (TURP), use in trans rectal prostate ultrasound, biopsy, and radiation treatment.

14. A method for using the medical device of claim 1, the method being directed to use in procedural environments, operatory and surgical procedures, ambulatory and out-patient procedures and unobtrusive normal routine living.

15. A plurality of medical devices in communication with at least one repository, the repository comprising at least one of a heat sink, a chemical reactor and a storage vessel, at least one of the plurality of medical devices comprising at least one of a cooling system and a material discharge system, wherein the at least one repository is positioned in at least one of intra body and outside the body.
